(19) United States
(12) Patent Application Publication
(10) Pub. No.: US 2022/0230730 A1
(43) Pub. Date: Jul. 21, 2022

(54) RECIPE RECOMMENDATION METHOD AND DEVICE, COMPUTING DEVICE AND STORAGE MEDIUM

(71) Applicant: BOE TECHNOLOGY GROUP CO., LTD., Beijing (CN)

(72) Inventors: Lu ZHANG, Beijing (CN); Zhenzhong ZHANG, Beijing (CN)

(21) Appl. No.: 17/452,934

(22) Filed: Oct. 29, 2021

(30) Foreign Application Priority Data
Jan. 18, 2021 (CN) 202110065106.X

(51) Int. Cl. G16H 20/60; A61B 5/11; A61B 5/0537; A61B 5/00

(52) U.S. Cl. CPC G16H 20/60; A61B 5/4866; A61B 5/0537; A61B 5/1118

(57) ABSTRACT

There are disclosed a recipe recommendation method and device, a computing device and a storage medium. The method includes: obtaining characteristic information of a user the characteristic information including static characteristic information and dynamic characteristic information of the user; determining a user requirement based on the characteristic information of the user; determining a recommended recipe based on the user requirement.

### RELATED APPLICATION

The present application claims the benefit of Chinese Patent Application No. 202110065106.X, filed on Jan. 18, 2021, the entire disclosures of which are incorporated herein by reference.

### TECHNICAL FIELD

The present disclosure generally relates to the field of intelligent recommendation, particularly to a recipe recommendation method and device, a computing device and a storage medium.

### BACKGROUND

With the gradual improvement of people's living standards, healthy diet has become a major theme in people's diet. In related technologies, recipe recommendation is usually based on users' emotions. However, with the increasing user requirements, there are one-sidedness and limitations in recommending recipes only based on users' emotions. For example, such a recipe recommendation method cannot meet the user requirements for personalization and refinement of recommended recipes, which seriously affects users' satisfaction with the recommended recipes.

### SUMMARY

In view of the above defects or shortcomings, it is desired to provide a recipe recommendation method and device, a computing device and a storage medium, so as to provide personalized, refined, healthy and diversified recipe recommendations for the user.

According to an aspect of the present disclosure, a recipe recommendation method is provided, comprising: obtaining characteristic information of a user, the characteristic information of the user comprising static characteristic information and dynamic characteristic information of the user; determining a user requirement based on the characteristic information of the user; determining a recommended recipe based on the user requirement.

In some embodiments, the static characteristic information of the user comprises attribute information of the user, the dynamic characteristic information of the user comprises a physical activity of the user within a predetermined time period, and the user requirement comprises an energy requirement of the user.

In some embodiments, the determining a user requirement based on the characteristic information of the user comprises: calculating a basal metabolic rate of the user based on the attribute information of the user; determining a physical activity level coefficient of the user based on the physical activity of the user within a predetermined time period; calculating the energy requirement of the user based on the basal metabolic rate and the physical activity level coefficient of the user.

In some embodiments, the determining a physical activity level coefficient of the user based on the physical activity of the user within a predetermined time period comprises: determining an exercise level of the user based on the physical activity of the user within a predetermined time period; determining the physical activity level coefficient of the user based on the exercise level of the user.

In some embodiments, the static characteristic information of the user further comprises taste related information of the user, the dynamic characteristic information of the user further comprises a physical state of the user, and the user requirement further comprises a taste requirement of the user.

In some embodiments, the determining a user requirement based on the characteristic information of the user further comprises: obtaining the taste requirement of the user based on the taste related information and the physical state of the user, wherein the taste requirement of the user comprises a taste preference and a taste taboo of the user; and the determining a recommended recipe based on the user requirement comprises: determining a recommended recipe based on at least one of the taste requirement and the energy requirement of the user.

In some embodiments, the determining a recommended recipe based on at least one of the taste requirement and the energy requirement of the user comprises: determining a candidate food based on the taste requirement of the user; generating a candidate recipe based on the candidate food; quantifying weights of candidate ingredients in the candidate recipe based on the energy requirement of the user; determining a recommended recipe based on the weights of candidate ingredients in the candidate recipe and the candidate recipe.

In some embodiments, the determining a candidate food based on the taste requirement of the user comprises: selecting an initial candidate food set from a predetermined food bank based on the taste taboo of the user; selecting a preferred food for the user from the initial candidate food set based on the taste preference of the user; determining, for each food in the initial candidate food set, a nutritional claim of the food based on a nutritional claim of main ingredients, a cooking method and a weight proportion of auxiliary ingredients of the food; calculating, for each food in the initial candidate food set, a similarity between the food and the preferred food based on the nutritional claim, the cooking method and the main ingredients of the food; selecting a candidate food from the initial candidate food set based on the similarity between each food in the initial candidate food set and the preferred food.

In some embodiments, the determining a candidate food based on the taste requirement of the user further comprises: prior to the selecting a candidate food from the initial candidate food set based on the similarity between each food in the initial candidate food set and the preferred food, updating the initial candidate food set based on the taste taboo and the nutritional claim of each food in the initial candidate food set.

In some embodiments, the nutritional claim comprises a quantifiable nutritional claim and a non-quantifiable nutritional claim; and the determining, for each food in the initial candidate food set, a nutritional claim of the food based on a nutritional claim of main ingredients, a cooking method and a weight proportion of auxiliary ingredients of the food comprises: determining whether the weight proportion of auxiliary ingredients in the food exceeds a preset threshold; in response to the weight proportion of auxiliary ingredients in the food exceeding the preset threshold, obtaining the quantifiable nutritional claim of the auxiliary ingredients and determining the nutritional claim of the food as a nutritional claim of the main ingredients and the quantifiable nutritional claim of the auxiliary ingredients; in response to the weight proportion of auxiliary ingredients in the food not exceeding the preset threshold, determining the nutritional claim of the food as a nutritional claim of the main ingredients.

In some embodiments, the generating a candidate recipe based on the candidate food comprises: obtaining a recipe combination strategy; generating a candidate recipe based on the recipe combination strategy and the candidate food.

In some embodiments, the quantifying weights of candidate ingredients in the candidate recipe based on the energy requirement of the user comprises: determining standard weights of nutrients required by the user according to preset energy supply percentages based on the energy requirement of the user, the nutrients comprising fat, protein and carbohydrate; obtaining a nutrient content of each candidate ingredient in the candidate recipe; calculating a weight of each candidate ingredient in the candidate recipe based on the standard weights of the nutrients and the nutrient content of each candidate ingredient in the candidate recipe.

In some embodiments, the calculating a weight of each candidate ingredient in the candidate recipe based on the standard weights of the nutrients and the nutrient content of each candidate ingredient in the candidate recipe comprises: calculating the weight of each candidate ingredient in the candidate recipe by solving the following nutrient weight equation set:

$$\{ \begin{matrix}{fat}_{1} * {x}_{1} + {fat}_{2} * {x}_{2} + . . . + {fat}_{m} * {x}_{m} = {Weight}_{f} \\ {protein}_{1} * {x}_{1} + {protein}_{2} * {x}_{2} + . . . + {protein}_{m} * {x}_{m} = {Weight}_{p} \\ \begin{matrix}{carbohydrate}_{1} * {x}_{1} + {carbohydrate}_{2} * {x}_{2} + . . . + \\ {carbohydrate}_{m} * {x}_{m} = {Weight}_{c}\end{matrix}\end{matrix}$$

wherein in is the number of the candidate ingredients in the candidate recipe, xᵢ is the weight of the ith ingredient, fatᵢ is the fat content of the ith ingredient, proteinᵢ is the protein content of the ith ingredient, carbohydrateᵢ is the carbohydrate content of the ith ingredient, wherein i=1, . . . , m; Weight_{f}, Weightₚ, Weight_{c} respectively represent standard weights of fat, protein, carbohydrate required by the user.

In some embodiments, the calculating the weight of each candidate ingredient in the candidate recipe by solving the following nutrient weight equation set comprises: obtaining a scoring function of the solution of the nutrient equation set by the following formula:

$$Score = \frac{\vert \overline{{W}_{f}} - {Weight}_{f} \vert}{{Weight}_{f}} + \frac{\vert \overline{{W}_{p}} - {Weight}_{p} \vert}{{Weight}_{p}} + \frac{\vert \overline{{W}_{c}} - {Weight}_{c} \vert}{{Weight}_{c}} ,$$

wherein Score represents the dependent variable of the scoring function, W̅_{f}, W̅ₚ, W̅_{c} are respectively defined by the following formula:

$$\{ \begin{matrix}\overline{{W}_{f}} = {fat}_{1} * {x}_{1} + {fat}_{2} * {x}_{2} + . . . + {fat}_{m} * {x}_{m} \\ \overline{{W}_{p}} = {protein}_{1} * {x}_{1} + {protein}_{2} * {x}_{2} + . . . + {protein}_{m} * {x}_{m} \\ \begin{matrix}\overline{{W}_{c}} = {carbohydrate}_{1} * {x}_{1} + {carbohydrate}_{2} * \\ {x}_{2} + . . . + {carbohydrate}_{m} * {x}_{m}\end{matrix}\end{matrix} ;$$

determining a value of the independent variable corresponding to the minimum value of the scoring function as an optimal solution of the nutrient equation set by means of an optimization algorithm.

In some embodiments, the obtaining characteristic information of a user comprises at least one of the following steps: collecting the characteristic information of the user through human-computer interaction; receiving the characteristic information of the user from at least one service platform; collecting the characteristic information of the user through at least one of face monitoring and physical state monitoring.

According to another aspect of the present disclosure, a recipe recommendation device is provided, comprising: an obtaining module configured to obtain characteristic information of a user, the characteristic information of the user comprising static characteristic information and dynamic characteristic information of the user; a determination module configured to determine a user requirement based on the characteristic information of the user; a recommendation module configured to determine a recommended recipe based on the user requirement.

According to a further aspect of the present disclosure, a computing device is provided, comprising: a processor; and a memory with instructions stored thereon, which instructions, when executed on the processor, cause the processor to carry out the recipe recommendation method according to some embodiments of the present disclosure.

According to yet another aspect of the present disclosure, a computer readable storage medium is provided, with computer readable instructions stored thereon which, when executed, carry out the recipe recommendation method according to some embodiments of the present disclosure.

In the recipe recommendation method according to some embodiments of the present disclosure, the user's various requirements (e.g., including taste requirement and energy requirement) on diet are obtained based on the static characteristic information and the dynamic characteristic information of the user, and the recommended recipe is determined based on the user requirements. Hence, in the recipe recommendation method, by comprehensively considering the user's multi-dimensional personal characteristic information (static characteristic information such as user attribute information and dynamic characteristic information such as physical activity and real-time physical state), the user's personalized and accurate dietary requirements are fully reflected, and by determining recipes based on various user requirements (such as subjective taste requirements and objective energy requirements), healthy, refined and diversified food or recipe recommendations are realized.

It should be appreciated that the contents described in this part are not intended to identify key or important features of the embodiments of the present application, nor are they used to limit the scope of the present application.

### BRIEF DESCRIPTION OF THE DRAWINGS

By reading the detailed description on the non-limiting embodiments made with reference to the following drawings, other features, purposes and advantages of the present application will become more obvious:

FIG. 1 shows an example application scene of a recipe recommendation method according to some embodiments of the present disclosure;

FIG. 2 shows an example architecture of a recipe recommendation system according to some embodiments of the present disclosure;

FIG. 3 shows a flow chart of a recipe recommendation method according to some embodiments of the present disclosure;

FIG. 4 shows a flow chart of a recipe recommendation method according to some embodiments of the present disclosure;

FIG. 5 shows a flow chart of a recipe recommendation method according to some embodiments of the present disclosure;

FIG. 6 shows a flow chart of a recipe recommendation method according to some embodiments of the present disclosure;

FIG. 7 shows a flow chart of a recipe recommendation method according to some embodiments of the present disclosure;

FIG. 8 shows a flow chart of a recipe recommendation method according to some embodiments of the present disclosure;

FIG. 9 shows a structural block diagram of a recipe recommendation device according to some embodiments of the present disclosure; and

FIG. 10 shows a structural block diagram of a computing device according to some embodiments of the present disclosure.

### DETAILED DESCRIPTION OF THE DISCLOSURE

The present application will be further described in detail below in combination with the accompanying drawings and embodiments. It could be appreciated that the specific embodiments described herein are only used to explain the relevant invention rather than to limit the invention.

It will be appreciated that although the terms such as first, second, third can be used herein for describing various elements, components and/or parts, these elements, components and/or parts should not be limited by these terms. These terms are only used to distinguish one element, component and/or part from another. Therefore, the first element, component or part discussed below can be called the second element, component or part without deviating from the teachings of the present disclosure.

The terms used herein are for the purpose of describing specific embodiments only and are not intended to limit the present disclosure. As used herein, the singular forms “a”, “one” and “the” intend also to include plural forms, unless otherwise instructed in the context clearly. It will be further understood that the terms “comprise” and/or “include”, when used in this Specification, specify the presence of the described features, entirety, steps, operations, elements and/or components, while not excluding presence of one or more other features, entirety, steps, operations, elements, components and/or groups thereof or adding one or more other features, entirety, steps, operations, elements, components and/or groups thereof. As used herein, the term “and/or” includes any and all combinations of one or more of the associated listed items.

Unless otherwise defined, all terms (including technical terms and scientific terms) used herein have the same meanings as generally understood by the ordinary skilled person in the art to which the present disclosure belongs. It will be further appreciated that terms such as those defined in commonly used dictionaries should be interpreted as having meanings consistent with their meanings in the relevant field and/or in the context of this Specification, and will not be interpreted in an idealized or overly formal sense unless explicitly defined herein. It should be noted that in the event of not conflicting, the embodiments in the present disclosure and the features in the embodiments can be combined with each other.

At present, the food or recipe recommendation schemes of related technologies often unilaterally consider the user's single characteristic information (such as the user's emotional state), which cannot meet the growing personalized and accurate requirements of users for food or recipe recommendation. Facing the above problems, the present disclosure proposes a recipe recommendation method, which comprehensively considers a variety of user's characteristic information (static characteristic information such as body attributes and dynamic characteristic information such as real-time physical state) to realize personalized, healthy, refined and diversified food or recipe recommendation.

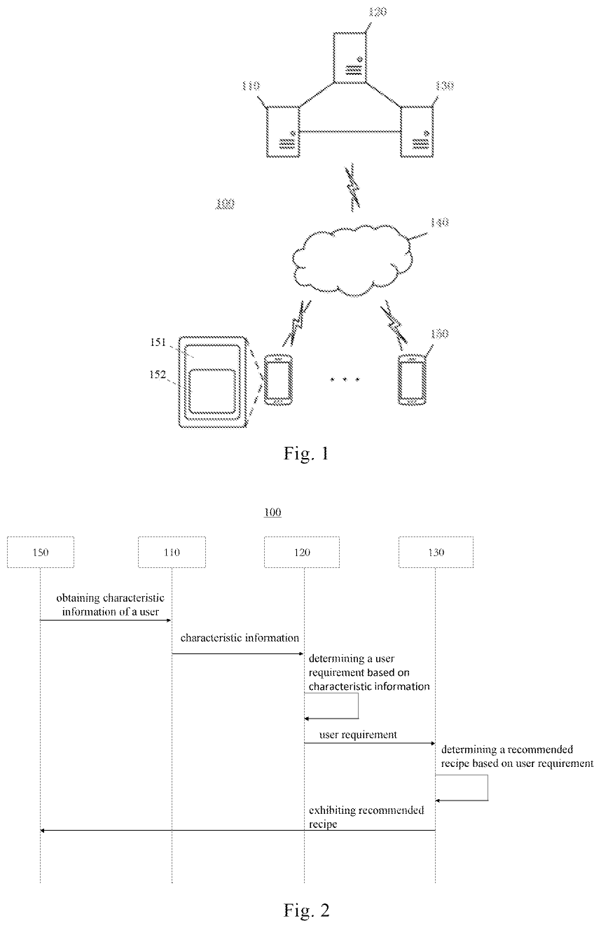

FIG. 1 shows an example application scene of a recipe recommendation method according to some embodiments of the present disclosure, i.e., a recipe recommendation system 100. As shown in FIG. 1, the recipe recommendation system 100 may comprise a user management server 110, a requirement management server 120, a recipe management server 130, and optionally comprises a network 140 and one or more terminal devices 150. In some embodiments, the recipe recommendation system 100 may be used to implement various methods described herein, such as a recipe recommendation method according to some embodiments of the present disclosure.

The user management server 110, the requirement management server 120 and the recipe management server 130 can store and run instructions capable of performing various method described herein, which respectively can be a single server or a server cluster or a distributed system, or can be a cloud server capable of providing basic cloud computing services such as cloud services, cloud databases, cloud computing, cloud functions, cloud storage, network services, cloud communication, middleware services, domain name services, safety services, CDN, and big data and artificial intelligence platform, etc., or any two or three of which can be the same server or the same server cluster. It should be appreciated that the servers mentioned herein are typically server computers with a large amount of memory and processor resources, however, other embodiments are also possible.

The network 140 can be a wireless network or a wired network using standard communication technologies and/or protocols. The example of the network 140 generally includes Internet, and may also be any other networks, for example, including but not limited to Local Area Network (LAN), Metropolitan Area Network (MAN), Wide Area Network (WAN), Personal Area Network (PAN), mobile communication network, dedicated network, or virtual dedicated network, or any combination of the above types of network. Each of the user management server 110, the requirement management server 120, the recipe management server 130 and one or more terminal devices 150 can include at least one communication interface (not shown) capable of performing communication via the network 140. Such communication interfaces can be one or more of the following: any type of network interfaces (e.g., network interface card (NIC)), a wired or wireless (such as IEEE 802.11 wireless LAN (WLAN)) interface, a World Interoperability for Microwave Access (Wi-MAX) interface, an Ethernet interface, a universal serial bus (USB) interface, a cellular network interface, a Bluetooth™ interface, a near field communication (NFC) interface, etc.

The terminal device 150 may be any type of mobile computing devices, including mobile computers (e.g., personal digital assistants (PDAs), laptops, notebooks, tablets, netbooks, etc.), mobile phones (e.g., cellular phones, smart phones, etc.), wearable computing devices (e.g., smart watches, head mounted devices, including smart glasses, etc.) or other types of mobile devices. In some embodiments, the terminal device 150 may also be a fixed computing device, such as a desktop computer, a game console, a smart TV, etc. In addition, when the recipe recommendation system 100 includes a plurality of terminal devices 150, the plurality of terminal devices 150 may be the same or different types of computing devices.

As shown in FIG. 1, the terminal device 150 may comprise a display screen 151 and a terminal application 152 that can interact with a terminal user via the display screen 151. The terminal device 150 may for example interact with one or more of the user management server 110, the requirement management server 120 and the recipe management server 130 via the network 140, e.g., transmitting data to or receiving data from it. The terminal application 152 may be a local application, a Web application, or an applet as a lightweight application (LiteApp, such as a mobile app or a WeChat applet). When the terminal application 152 is a local application to be installed, the terminal application 152 may be installed in the terminal device 150. When the terminal application 152 is a Web application, the terminal application 152 can be accessed through a browser. When the terminal application 152 is an applet, the terminal application 152 can be opened directly on the user terminal 150 by searching the relevant information of the terminal application 152 (such as the name of the terminal application 152), scanning the graphic code of the terminal application 152 (such as bar code, QR code, etc.) and so on, without installing the terminal application 152.

FIG. 2 shows an example architecture of a recipe recommendation system 100 according to some embodiments of the present disclosure. The operating principle of the recipe recommendation system 100 will be briefly described below with reference to the example architecture as shown in FIG. 2.

As shown in FIG. 2, in the recipe recommendation system 100, the user management server 110 is configured to obtain characteristic information of a user, wherein the characteristic information includes static characteristic information and dynamic characteristic information of the user. As shown in FIG. 2, the characteristic information of the user can be obtained in two ways: one is active, that is, performing face monitoring and/or physical state monitoring (such as blood pressure monitoring) on the user through the terminal device 150, or receiving the user's input motion information through the terminal application such as AI health assistant to collect the user's real-time dynamic characteristic information (such as real-time physical state); the second is passive, that is, in response to a user request (including, for example, a recipe recommendation request or a client login request) received from the terminal device 150, obtaining the static feature information (such as body attribute information or taste related information) registered or input by the user. In some embodiments, the user preference information obtained or determined by the user management server 110 can be properly processed to form a user preference information data pipeline and be written into to a relay message queue, which can be read by other servers or other devices.

As shown in FIG. 2, in the recipe recommendation system 100, the requirement management server 120 is configured to determine a user requirement based on the characteristic information of the user. For example, as shown in FIG. 2, the requirement management server 120 may directly receive the characteristic information data or request data pipeline including the characteristic information from the user management server 110; or the requirement management server 120 can read the characteristic information data pipeline from the relay message queue to obtain the characteristic information of the user.

As shown in FIG. 2, in the recipe recommendation system 100, the recipe management server 130 is configured to determine a recommended recipe based on the user requirement. In some embodiments, as shown in FIG. 2, the recipe management server 130 can receive a user requirement message from the requirement management server 120 and recommend a corresponding recipe in response to the message. Optionally, as shown in FIG. 2, the recipe management server 130 can exhibit the recommended recipe to the user through the terminal device 150, for example, sending the recommended recipe and the following command to the terminal device 150: displaying the recommended recipe on the display 151 of the terminal device 150.

It should be appreciated that although the user management server 110, the requirement management server 120, the recipe management server 130 and the terminal device 150 are shown and described as separate structures herein, they may also be different components of the same computing device, wherein the user management server 110, the requirement management server 120, and the recipe management server 130 can provide background computing function, while the terminal device 150 can provide foreground display function and other optional functions, such as characteristic information collection function.

In order to further explain the technical solution provided by the embodiment of the present application, it will be described in detail below in combination with the accompanying drawings and specific embodiments. Although the embodiments of the present application provide the method operation instruction steps shown in the following embodiments or the accompanying drawings, the method may include more or fewer operation instruction steps based on conventional or non-inventive effort. Among the steps in which there is no necessary causal relationship logically, the execution sequence of these steps is not limited to the execution sequence provided by the embodiment of the present application. The method may be executed sequentially or in parallel according to the method shown in the embodiment or the accompanying drawings in the actual processing process or when executed by the device.

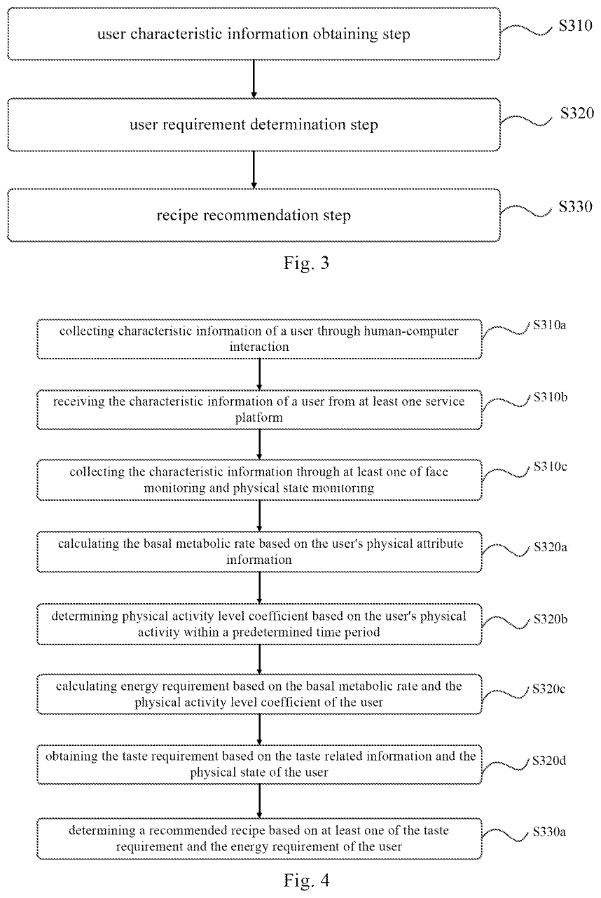

FIG. 3 shows a flow chart of a recipe recommendation method according to some embodiments of the present disclosure. The recipe recommendation method according to some embodiments of the present disclosure as shown in FIG. 3 can be performed in, for example, the recipe recommendation system 100 shown in FIG. 1 and FIG. 2.

As shown in FIG. 3, the recipe recommendation method may comprise the steps of: S310, user characteristic information obtaining step; S320, user requirement determination step; and S330, recipe recommendation step. The steps S310-S330 as shown in FIG. 3 will be introduced below in detail.

In the user characteristic information obtaining step S310, obtaining characteristic information of a user. The characteristic information of the user may include static characteristic information and dynamic characteristic information of the user.

According to the concept of the present disclosure, in order to realize the accuracy and personalization of recipe recommendation, a variety of factors can be comprehensively considered, such as user's personalized requirements, including personalized energy or nutrition requirement and personalized taste requirement. These personalized requirements can be determined based on the characteristic information of the user. The characteristic information of the user herein may refer to the user's personal information related to the recipe recommendation for the user or affecting the user's diet, such as taste preference, physical state, health status, etc. Generally speaking, the characteristic information of the user can include relatively stable (basically unchanged or with a low change frequency) characteristic information, that is, static characteristic information; it can also include dynamically changing characteristic information, that is, dynamic characteristic information.

In some embodiments, the static characteristic information of the user can include user's attribute information, such as the user's gender, height, weight, age, etc.; or it can further include taste related information, such as ethnicity, region, taste, taboo food, historical data of physical health (such as previous medical history or periodic (e.g. annual) physical examination results). In some embodiments, the dynamic characteristic information of the user can include: physical activity of the user within a predetermined time period, such as weekly exercise and/or physical work related to physical activity; and optionally, it can also include the user's physical state, such as the user's real-time mental state (including emotional and psychological state) and physiological state (such as real-time physiological index) or real-time health data (such as getting inflamed, catching a cold, having a fever, etc.).

In some embodiments, the physical activity of the user within a predetermined time period can be measured by the user's exercise level. The exercise level can be determined according to the data such as motion type, motion time, motion frequency specifically. The predetermined time period can be a preset time period, such as a day, a week, a month, etc., or other time periods. For example, the real-time updated physical activity can be obtained every predetermined time period, and the exercise level can be determined according to the latest information, so as to determine the user's energy requirement.

In some embodiments, the static characteristic information (e.g., including body attribute information and taste related information) can be obtained through an interactive interface on the terminal device. For example, in response to a user request (e.g., including a recipe recommendation request or a client login request) received from the terminal device 150, static characteristic information (such as body attribute information or taste related information) registered or input by the user is obtained. In addition, the static characteristic information can also be obtained through other channels, for example, the user management server can (with the permission of the user) be connected to the physical examination system to capture the physical examination results of the user to obtain the historical data of the user's health status. In some embodiments, the dynamic characteristic information (such as real-time physical state or physical activity) can be collected through the terminal device and/or other information collection devices (such as human physiological information collection device) bound to the terminal device or user account.

In the user requirement determination step S320, determining a user requirement based on the characteristic information of the user.

Based on the concept of the present disclosure, after the characteristic information of the user is obtained, the user's (personalized) requirement can be obtained based on the characteristic information. The user requirement may include personalized energy or nutrition requirement and personalized taste requirement. In the user's personalized requirement for recipe recommendation, the user's energy requirement can depend on the body attribute information in the user's static characteristic information and the physical activity in the user's dynamic characteristic information, while the user's taste requirement is related to, for example, the taste related information in the static characteristic information and the physical state in the dynamic characteristic information, etc.

It should be noted that the user's energy requirement refers to the energy required by the user to maintain the user's health within a certain period of time (e.g. every day, every week). The user's energy requirement can be calculated according to the user's physical attributes (such as height, weight, age, gender, etc.) and physical activity (such as physical activity related to daily (such as weekly) exercise or working state, especially physical work) within a predetermined time period. The user's energy requirement is an important consideration for recipe recommendation, because the determination of the user's energy requirement is equivalent to giving an energy standard of the recommended recipe, which can prevent excessive nutrition of the user caused by providing excessive energy during recipe recommendation, or avoid the user's fatigue even unable to work and live normally due to too little energy provided by the recommended recipe.

On the other hand, the taste requirement can include taste preference and taste taboo, which can be determined based on factors such as taste related information in the static characteristic information and physical state in the dynamic characteristic information. The taste requirement is also an important factor to be considered in diet or recipe recommendation. It can fully consider the impact of users' personal subjective preferences and objective health conditions on diet, so as to improve user experience and achieve the purpose of personalized and refined recipe recommendation.

In the recipe recommendation step S330, determining a recommended recipe based on the user requirement.

As mentioned above, the user requirement can include energy requirement and taste requirement. Therefore, when determining the recommended recipe, the recommended recipe can be generated based on at least one of the energy requirement and the taste requirement. For example, for a specific user (such as a healthy user, a user without taste preference or a user insensitive to taste), a recommended recipe can be generated solely based on the energy requirement, that is, as long as the recipe meets the energy requirement, it can be recommended to the user; for example, recipes can occasionally be recommended to some users who do not have high energy requirements only based on the taste requirement.

In some embodiments, both energy requirement and taste requirement can be considered to recommend recipes, so as to take into account of both the objective physiological requirement and the subjective taste requirement and improve the user experience. For example, the step S330 may comprise: firstly, determining candidate food based on the taste requirement, then generating a candidate recipe based on the candidate food, and then generating a recommended recipe based on the energy requirement and the candidate recipe.

In the recipe recommendation method according to some embodiments of the present disclosure, the user's various requirements (e.g., including taste requirement and energy requirement) on diet are obtained based on the static characteristic information and dynamic characteristic information of the user, and the recommended recipe is determined based on the user requirements. Hence, in the recipe recommendation method, by comprehensively considering the user's multi-dimensional personal characteristic information (static characteristic information such as user attribute information and dynamic characteristic information such as physical activity and real-time physical state), the user's personalized and accurate dietary requirements are fully reflected, and by determining recipes based on various user requirements (such as subjective taste requirements and objective energy requirements), healthy, refined and diversified food or recipe recommendations are realized.

FIG. 4 shows a flow chart of a recipe recommendation method according to some embodiments of the present disclosure.

As shown in FIG. 4, the user characteristic information obtaining step S310 in FIG. 3 may comprise at least one of the following steps:

S310a, collecting characteristic information of a user through human-computer interaction;

S310b, receiving the characteristic information of the user from at least one service platform;

S310c, collecting the characteristic information of the user through at least one of face monitoring and physical state monitoring.

In some embodiments, as shown in S310a, the characteristic information of the user can be collected through human-computer interaction. The static characteristic information (e.g., including physical attribute information and taste related information) for example can be obtained through an interactive interface on the terminal device. For example, when the user initially uses the application (such as mobile app) on the terminal device, the static characteristic information is input through the personal information registration interface. At this time, the user management server can directly obtain these information of relevant users from the database used to store the registration information. Optionally, the user management server can also receive the static characteristic information input by the user in real time directly through the interactive interface of the terminal device when recommending recipes. It should also be understood that the interactive interface of the terminal device can be provided by an application (such as AI health assistant) loaded in the terminal device. When using the terminal device, the user starts the application by touching, and the application invokes and displays the interactive interface that can receive the user's characteristic information. Specifically, when obtaining the characteristic information, the application of the terminal device calls the information editing interface, which includes information collection control, and the user can input the user's characteristic information (such as static characteristic information or dynamic characteristic information) on the collection control of the editing interface. The application monitors the collection control to obtain the information input by the user.

In addition, as shown in S310b, the characteristic information can of the user also be obtained through a service platform (e.g., a database in which the characteristic information of the user is stored in advance). The service platform can be for example, the built-in data platform of the exercise bracelet, the data platform corresponding to the physical examination system, etc. In this way, the user's exercise information can be obtained through the exercise bracelet, and the user's physical examination results and health information can be obtained through the physical examination system. For example, the user management server can (with the permission of the user) be connected to the physical examination system to capture the user's physical examination results to obtain the historical data of the user's health status.

In some embodiments, as shown in S310c, the characteristic information of the user can be collected through at least one of face monitoring and physical state monitoring. The dynamic characteristic information (such as the real time physical state or physical activity) can be collected through the terminal device and/or other information collection devices (such as human physiological information collection device) bound to the terminal device or user account. For example, the terminal device obtains the user's real-time physical physiological state (such as heartbeat, blood pressure, etc.) or exercise condition (such as exercise volume (such as walking steps, jogging mileage), calorie consumption, etc.) through data transmission with the exercise wristband or bracelet worn by the user. For example, the upload cycle of information collection can be preset, and the obtained information such as user physical activity can be directly sent to the user management server or indirectly sent to the user management server through the terminal device according to the predetermined upload cycle. For example, the user's facial expression can be photographed and recognized by the camera to obtain the user's real-time physical state, such as real-time health. For example, it can be identified whether there are abnormalities (furuncle, aphtha, etc.) caused by inflammation on the user's face. When the corresponding abnormalities are identified, it can be considered that the user has inflammation symptoms. Optionally, technologies such as face monitoring and image recognition can also be used to directly obtain or predict the static characteristic information of the user, such as gender, height, weight, age and other user attribute information.

As shown in FIG. 4, the user requirement determination step S320 in FIG. 3 can include S320a-S320c.

As mentioned above, the user requirement can include energy requirement and taste requirement, wherein the energy requirement can be calculated based on the user's physical attributes and physical activities within a predetermined time period. Specifically, first, the basal metabolic rate needs to be calculated based on the physical attribute, then the physical activity level coefficient is determined based on the physical activity, and finally the user's energy requirement is obtained by multiplying the basal metabolic rate and the physical activity level coefficient. The steps S320a-S320c for determining the energy requirement will be introduced below in detail.

At step S320a, calculating a basal metabolic rate based on the user's physical attribute information.

In some embodiments, the basal metabolic rate of the user can be calculated by the following formula (1) based on the user's physical attribute information (including height, weight, age and gender):

$$\{ \begin{matrix}{BMR}_{W} = 65 + ( 9.6 * weight ) + ( 1.8 * height ) - ( 4.7 * age ) \\ {BMR}_{M} = 66 + ( 13.7 * weight ) + ( 5 * height ) - ( 6.8 * age )\end{matrix} \qquad ( 1 )$$

In the above formula (1), BMR_{W} represents the female basal metabolic rate, BMR_{M} represents the male basal metabolic rate, the unit of weight is kilogram (kg), the unit of height is metre (m), the unit of age is year, the unit of BMR_{W} and BMR_{M} is kilocalorie (kcal).

At step S320b, determining a physical activity level coefficient of the user based on the physical activity of the user within a predetermined time period.

The physical activity level coefficient φ is a coefficient related to people's physical activity (e.g. exercise/working state) within a period of time, in which the period of time can be a preset period of time, that is, a predetermined period of time, such as a week. If a person has a large amount of exercise or heavy physical work in a week, the physical activity level coefficient φ will be relatively large. If a person has little exercise or light physical work in a week, the physical activity level coefficient φ will be relatively small. The variation range of φ can be [1.2, 1.9].

In some embodiments, the step S320b may comprise: determining, based on the user's physical activity within a predetermined time period, user's exercise level; determining, based on the user's exercise level, user's physical activity level coefficient.

In some embodiments, the user's physical activity within a predetermined period of time can be measured by the user's exercise level. The exercise level can be determined based on the specific physical activity, for example, it can be determined and divided according to the actual exercise type, exercise time, exercise frequency and other data in a week. For example, for those who sit in the office or classroom for a long time and have little or no exercise at all, the exercise level is determined as level 1; for those who exercise occasionally or take a walk, go shopping or go for an outing in the suburbs, and exercise a little about 1-3 times a week, the exercise level is determined as level 2; for those who have the habit of continuous exercise or go to the gym about 3-5 times a week, the level of exercise is determined as level 3; for those who love sports, exercise 6-7 times a week, or have a large workload, the level of exercise is determined as level 4; for those who need a lot of (physical) labor for work or life and consume considerable energy, the exercise level is determined as level 5.

In some embodiments, the exercise level can also be determined by preset analysis functions, analysis rules and/or trained in-depth learning models according to the physical activities within the predetermined time (user exercise frequency, exercise time and exercise type, etc.), which is not specifically limited by the present application here.

In some embodiments, there is a mapping relationship between the exercise level and the physical activity level coefficient, that is, they can be in one-to-one correspondence, so the physical activity level coefficient can be obtained directly based on the exercise level. For example, a mapping relationship table between the exercise level and the physical activity level coefficient can be preset and stored, so that the physical activity level coefficient can be directly queried from the preset mapping relationship table after the exercise level corresponding to the physical activity is obtained. Table 1 shows an example of a direct mapping relationship table between exercise level and physical activity level coefficient. It can be seen from Table 1 that the physical activity level coefficient corresponding to exercise level 1 is φ=1.2, the physical activity level coefficient corresponding to exercise level 2 is φ=1.375, the physical activity level coefficient corresponding to exercise level 3 is φ=1.55, the physical activity level coefficient corresponding to exercise level 4 is φ=1.7, the physical activity level coefficient corresponding to exercise level 5 is φ=1.9.

TABLE 1

| Exercise level | 1 | 2 | 3 | 4 | 5 |
|---|---|---|---|---|---|
| Physical activity level coefficient (φ) | 1.2 | 1.375 | 1.55 | 1.7 | 1.9 |

It should be noted that since people's daily physical activities, such as exercise/working state, are dynamically changing, the physical activity level coefficient φ determined by obtaining the user's physical activities within a predetermined time period in real time will also be dynamically adjusted, thus it is determined that the user's energy requirement is also dynamically changing, so that the recommended recipes obtained can more accurately adapt to the user's dynamically changing physical activities.

At step S320c, calculating the use's energy requirement based on the user's basal metabolic rate and physical activity level coefficient.

In some embodiments, the user's energy requirement Energy_{T} can be obtained based on the user's basal metabolic rate BMR (which represents the general term of BMR_{W} and BMR_{M}) and the physical activity level coefficient φ through the following formula (2):

Energy_{T}=BMR*φ (2)

The currently obtained energy requirement Energy_{T} can be a total energy requirement of the user in a day. When making accurate recipe recommendations, it is also necessary to further determine the energy requirement corresponding to each meal of the user's three meals in a day. Then, the corresponding recommended recipe can be determined based on the energy requirement of each meal.

As shown in FIG. 4, the user requirement determination step S320 in FIG. 3 can further comprise:

S320d, obtaining a taste requirement of the user based on the taste related information and the physical state of the user.

In some embodiments, the taste requirement can include taste preference and taste taboo. The taste preference can refer to the user's subjective preference for taste or food or taste characteristics or food caused by objective factors, which can be determined by taste related information (such as preference, ethnicity, region, historical data of health, etc.). For example, ethnic or regional factors can form inherent taste characteristics, such as spicy taste characteristics formed due to humid climate in Sichuan Province, Hunan Province and other places of China. The taste taboo refers to the exclusion of specific tastes or foods formed due to subjective and objective factors, which can be determined by the user's health status. For example, physical health can include physical health historical data and real-time data. The real-time health data may include user's real-time physical state in the dynamic characteristic information and the health historical data such as chronic diseases in the taste related information (in the static characteristic information of the user). For example, three-high (i.e., Hyperlipidemia, hypertension, hyperglycemia) patients (historical data) try to reduce oil and salt to reduce the burden of blood vessels; while for real-time abnormal health status such as inflammation, users should avoid spicy and irritating food.

As shown in FIG. 4, the recipe recommendation step S330 in FIG. 3 may comprise:

S330a, determining a recommended recipe based on at least one of the taste requirement and the energy requirement of the user.

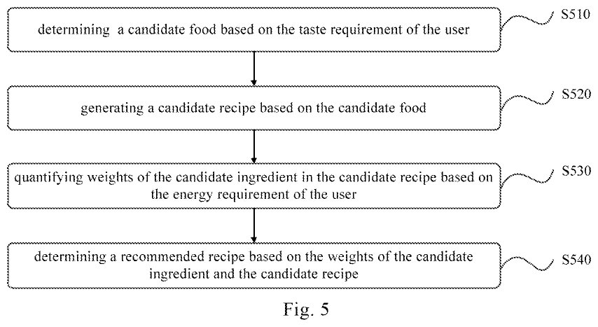

The user requirement can include energy requirement and taste requirement. Therefore, when determining the recommended recipe, the recommended recipe can be generated based on at least one of the energy requirement and the taste requirement. For example, for a specific user (such as a healthy user, a user without taste preference or a user insensitive to taste), a recommended recipe can be generated solely based on the energy requirement, that is, as long as the recipe meets the energy requirement, it can be recommended to the user; for example, recipes can occasionally be recommended to some users who do not have high energy requirements only based on the taste requirement. In some embodiments, both energy requirement and taste requirement can be considered to recommend recipes, so as to take into account the objective physiological requirements and subjective taste requirements and improve the user experience. The specific process of recommending recipes according to energy requirement and taste requirement can be seen in the example process as shown in FIG. 5.

FIG. 5 shows a flow chart of a recipe recommendation method according to some embodiments of the present disclosure.

As shown in FIG. 5, the step of S330a (determining a recommended recipe based on at least one of the taste requirement and the energy requirement of the user) as shown in FIG. 4 may comprise S510-S540.

At S510, determining a candidate food based on the taste requirement of the user.

When selecting candidate foods, one can first consider the taste taboos formed by the user's health information (for example, including real-time health data and/or historical data). For example, if the user has a history of diabetes, and the taste taboo includes avoiding foods with high sugar content, the selected candidate foods should not contain high sugar or high glycemic index foods. For another example, if it is detected that the user's real-time health condition is abnormal (e.g. getting inflamed), the taste taboo can be determined according to the specific abnormal condition. Secondly, the taste preference caused by the user's subjective or objective factors (such as region, habit, etc.) can also be considered, so as to determine the candidate food according to the taste preference after excluding the taste taboo food.

At step S520, generating a candidate recipe based on the candidate food.

After the candidate food is obtained, a candidate recipe can be generated based on the candidate food. For example, candidate recipes can be obtained directly based on candidate foods, personal eating habits or the eating habits of most people in big data. In some embodiments, first, a recipe combination strategy can be obtained or set, and then a candidate recipe can be generated according to the recipe combination strategy and the candidate food.

At step S530, quantifying weights of candidate ingredients in the candidate recipe based on the energy requirement of the user.

After the candidate recipe is obtained, it is also necessary to further convert it into the standard intake (weight) of various nutrients in food or ingredients according to the user's energy (or heat), and then calculate the specific weight of various candidate ingredients or foods in the candidate recipe according to the standard intake (weight) of nutrients, so as to realize refined recipe recommendation.

For example, when the daily total energy required has been calculated, the energy required for each meal can be determined based on the energy distribution proportion of three meals in a day; then, the daily energy required can be divided into the energy provided by nutrients according to the energy supply percentage of various nutrients in the diet. In this way, the energy provided by nutrients can be converted into the standard intake (weight) of corresponding nutrients according to the standard energy supply value (calories) of nutrients, so as to calculate the weight of ingredients based on the weight proportion of nutrients in various ingredients in the candidate recipes.

At step S540, determining a recommended recipe based on the weights of the candidate ingredients in the candidate recipe and the candidate recipe.

After the weight of various ingredients in the candidate recipe is obtained, the candidate recipe together with the corresponding weights of various ingredients can be taken as the recommended recipe. Optionally, after the weights of various ingredients in the candidate recipe is obtained, it can be further determined that whether the weight combination of ingredients in the candidate recipe composed of the weights of these ingredients is within a weight threshold range. If the weight combination of ingredients is within the weight threshold range, perform step S540 (that is, taking the candidate recipe together with the corresponding weights of various ingredients as the recommended recipe). If the weight combination of ingredients is not within the weight threshold range, the candidate food can be selected again from the candidate food list to replace the food in the original candidate recipe to form a new candidate recipe, and calculate a new weight combination of ingredients for the new candidate recipe until the weight threshold can be met.

It should also be understood that the final recommended recipe may include one or more recipes. Because the user's (static and dynamic) characteristic information includes various contents (such as physical health, taste related information, attribute information, physical activity, etc.), the resulting user requirements are also various. Therefore, the recommended recipe finally determined according to the user requirement can also be multiple recipes to provide more choices for the user, and for the convenience of the user to choose recipes therefrom which are more in line with the current requirement, so as to improve the user experience.

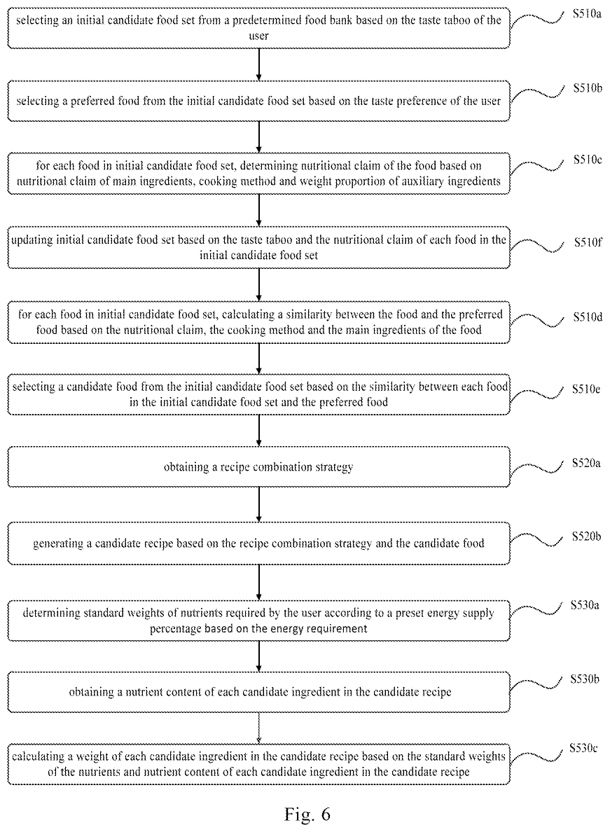

FIG. 6 shows an example flow chart of respective steps in the recipe recommendation method according to some embodiments of the present disclosure as shown in FIG. 5.

As shown in FIG. 6, the step S510 (determining a candidate food based on the taste requirement of the user) as shown in FIG. 5 can comprise S510a-S510e.

At S510a, selecting an initial candidate food set from the predetermined food bank based on the taste taboo of the user. When determining the candidate food, firstly, as shown in S510a, the taste taboo food can be removed from the given food bank according to the user's taste taboo, so as to screen out the initial candidate food set. The taste taboo includes taboo foods. Taboo foods can be ingredients that the user does not like. For example, if the user does not like to eat coriander, the taboo foods in the stored personality characteristic parameters can be coriander. On the other hand, taste taboos can also include inedible foods due to physical health.

At S510b, selecting a preferred food for the user from the initial candidate food set based on the taste preference of the user. It should be noted that the taste preference can be the pre-stored food that meets the user's taste preference, wherein the taste preference includes the food that the user likes. For example, if the user likes sweets, the taste preferences in the stored personality characteristic parameters can be Sweet and Sour Spare Ribs, Sweet and Sour Lotus Roots, etc., if the user likes spicy food, the taste preferences in the stored personality characteristic parameters can be Maoxuewang, Poached spicy slices of pork, etc.

At S510c, for each food in the initial candidate food set, determining a nutritional claim of the food based on a nutritional claim of main ingredients, a cooking method and a weight proportion of auxiliary ingredients of each food in the initial candidate food set.

It should be noted that the nutritional claim is a description and statement of the nutritional characteristics of food, such as energy level and protein content level, as specified in GB 28050 General Rules for Nutritional Labeling of Prepackaged Food in National Food Safety Standards. Specifically, nutritional claims can be divided into quantifiable and non-quantifiable nutritional claims. The quantifiable nutritional claims include, for example, high protein, high fat, high cholesterol, etc., the quantifiable nutritional claims also include, for example, the nutrient content of ingredients/food per unit weight, and the non-quantifiable nutritional claims include the properties of ingredients/food, such as spicy stimulation, non-digestibility, coldness, dryness, etc. The common nutritional claims include: high carbohydrate, high glycemic index, high fat, high cholesterol, high purine, high potassium, high sodium, dietary fiber, high vitamin C, etc. For example, non-quantifiable nutritional claims can be obtained according to the properties of food (e.g. according to the theory of traditional Chinese Medicine). Then, an ingredient bank is built according to various ingredients and their nutritional claims. Ingredients include main ingredients and auxiliary ingredients, and auxiliary ingredients include oil, salt, sugar, etc. Then the nutritional claim of the food is obtained. For example, the nutritional claim of the main ingredients can be taken as the nutritional claim of the food. For example, the nutritional claim of the food can also be determined according to the proportion of the main ingredients and the auxiliary ingredients. For example, the nutritional claim of food can also be determined using the cooking method, wherein the cooking method mainly includes steaming, boiling, cold mixing, frying, oil frying, roasting, etc.

The nutritional claim of the main ingredients, the cooking method and the weight proportion of the auxiliary ingredients of food can be obtained from the ingredient bank containing each ingredient and its nutritional claim, and each ingredient corresponds to at least one nutritional claim. The nutritional claims include quantifiable nutritional claims and non-quantifiable nutritional claims. For example, a list of ingredients corresponding to any food and the weight of each ingredient in the list of ingredients, as well as the main ingredients and auxiliary ingredients can be obtained from the ingredient bank, and the weight proportion of the main ingredients and auxiliary ingredients in the food can be determined according to the weight corresponding to each ingredient.

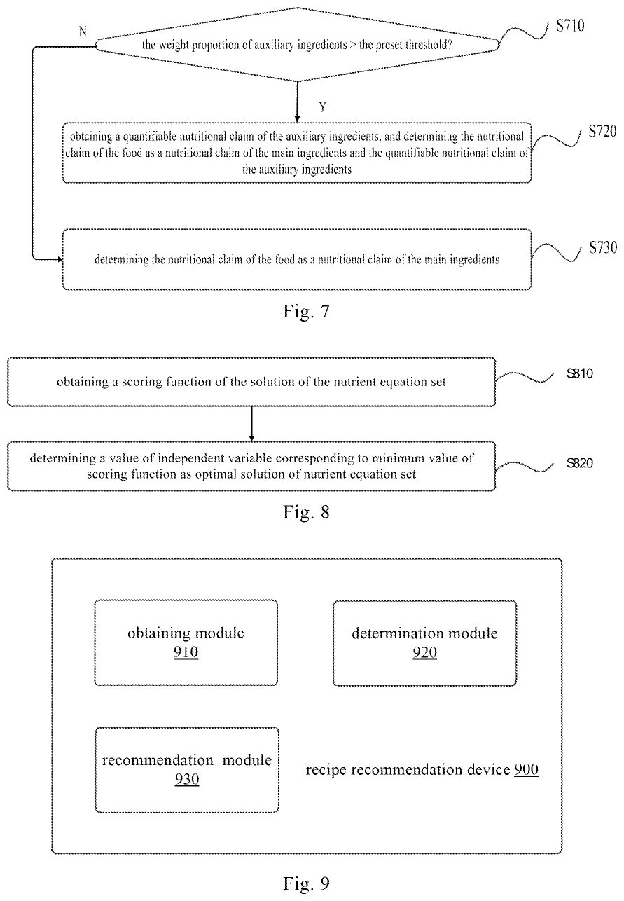

FIG. 7 shows an example flow chart of the step S510c. As shown in FIG. 7, in some embodiments, the step S510c (determining a nutritional claim of the food based on a nutritional claim of main ingredients, a cooking method and a weight proportion of auxiliary ingredients of each food in the initial candidate food set) may comprise:

S710, determining whether the weight proportion of auxiliary ingredients in the food exceeds a preset threshold;

S720, in response to the weight proportion of auxiliary ingredients in the food exceeding the preset threshold, obtaining a quantifiable nutritional claim of the auxiliary ingredients, and determining the nutritional claim of the food as a nutritional claim of the main ingredients and a quantifiable nutritional claim of the auxiliary ingredients;

S730, in response to the weight proportion of auxiliary ingredients in the food not exceeding the preset threshold, determining the nutritional claim of the food as a nutritional claim of the main ingredients.

As shown in S730, when the weight proportion of auxiliary ingredients in food is less than or equal to the preset threshold, the nutritional claim of main ingredients can be directly taken as the nutritional claim of the food. As shown in S720, when the weight proportion of auxiliary ingredients in food is greater than the preset threshold, the quantifiable nutritional claims corresponding to auxiliary ingredients, such as high fat, high sodium, high carbohydrate and high glycemic index, are further obtained. For example, when the weight of salt in the auxiliary ingredients is greater than the preset threshold, the quantifiable nutritional claim obtained is high sodium. Then, the nutritional claims corresponding to the main ingredients and the quantifiable nutritional claims corresponding to the auxiliary ingredients are taken as the nutritional claims of the food, wherein, the preset threshold can be ⅓˜¼. For example, when a food is composed of a single ingredient and does not contain auxiliary ingredients (i.e. the proportion of auxiliary ingredients is 0), the nutritional claim of the food is exactly the nutritional claim of the ingredient. For example, when the cooking method is frying or oil frying, the nutritional claim of the food can also include quantifiable nutritional claims such as high fat.

For example, the ingredients of the mashed eggplant with minced garlic are: 200 g eggplant, 10 g salad oil, 5 g pepper, 2 cloves of garlic, 0.5 tsp salt and 1 bundle of coriander, wherein, the eggplant is the main ingredient, the weight proportion of the salad oil and the salt is not high, there is no sugar, and the cooking method is cold mixing. The nutritional claim of the eggplant is {containing dietary fiber and cold}. After calculation, the proportion of auxiliary ingredients is less than ⅓, so the nutritional claim of the mashed eggplant with minced garlic is {containing dietary fiber and cold}.

At step S510d, for each food in the initial candidate food set, determining a similarity between the food and the preferred food based on the nutritional claim, the cooking method and the main ingredients of the food.

In some embodiments, the similarity between the food in the candidate food set to be selected and the preferred food involves three aspects: main ingredients, cooking methods and nutritional claims. Therefore, the similarity Sim₁ between the food to be selected and the preferred food in terms of main ingredients, the similarity Sim₂ in terms of cooking methods and the similarity Sim₃ in terms of nutritional claims can be calculated respectively; then the similarities of the three aspects are added to obtain the similarity Sim between the food to be selected and the preferred food, as shown in formula (3) below:

Sim=Sim₁+Sim₂+Sim₃ (3).

Optionally, the cosine algorithm can be used to calculate the similarities of the main ingredients, cooking methods and nutritional claims between the food to be selected and the preferred food, so as to obtain the similarity between the food to be selected and the preferred food. For example, the first word vector of the main ingredient information corresponding to the food to be selected and the second word vector of the main ingredient information corresponding to the preferred food can be obtained respectively, and then the cosine value between the first word vector and the second word vector can be calculated, and the obtained cosine value can be used as the main ingredient similarity between the food to be selected and the preferred food, and so on, using the third word vector corresponding to the cooking method of the food to be selected and the fourth word vector of the cooking method of the preferred food to obtain the cooking method similarity between the food to be selected and the preferred food; using the fifth word vector corresponding to the nutritional claim of the food to be selected and the sixth word vector corresponding to the nutritional claim of the taste preferred food to obtain the nutritional claim similarity between the food to be selected and the preferred food.

For example, there are main ingredients such as grain and potato, fruits and vegetables, meat, milk and eggs. The cooking methods mainly include steaming, boiling, cold mixing, frying, oil frying, roasting, etc. For example, assuming that the user's preferred food is Sweet and Sour Spare Ribs, the similarity between the mashed eggplant with minced garlic and Sweet and Sour Spare Ribs can be calculated to obtain the similarity value between the mashed eggplant with minced garlic and Sweet and Sour Spare Ribs.

At step S510e, selecting a candidate food from the initial candidate food set based on the similarity between each food in the initial candidate food set and the preferred food.

In some embodiments, after calculating, for each food to be selected in the initial candidate food set, the similarity between the food to be selected and the preferred food, a similarity list can be generated, including the mapping relationship between each food to be selected and the corresponding similarity. Afterwards, the similarity list is sorted in descending order of similarity, and then M foods to be selected with the highest similarity are selected, wherein M can be a positive integer greater than or equal to 1, such as 3 or 5.

Table 2 shows an example of a list of similarity between each food to be selected in the initial candidate food set and the preferred food. As shown in Table 2, the preferred food is Poached spicy slices of pork, which can also be used as food to be selected. Of course, the similarity is 100%. Alternatively, the preferred food may not be used as the food to be selected. As shown in Table 2, other foods to be selected and their similarities with the Poached spicy slices of pork are respectively: boiled fish being 98%, Maoxuewang being 97%, fish head with chopped pepper being 92%, and greedy frog being 88%. In this way, if M is 2, and if the preferred food Poached spicy slices of pork is not used as the food to be selected, the candidate foods will be boiled fish and Maoxuewang; If M is 3, the candidate foods will be boiled fish, Maoxuewang and fish head with chopped pepper.

TABLE 2

| | Food to be selected | | | | |
|---|---|---|---|---|---|
| | | | Fish head with | | Poached spicy |
| | Boiled fish | Maoxuewang | chopped pepper | Greedy frog | slices of pork |
| Similarity | 98% | 97% | 92% | 88% | 100% |

It should be understood that taking the food with the highest similarity as the candidate food can make the candidate food meet the user's taste preference as much as possible, and effectively avoid the recommended food affecting the user's dining experience because it does not meet the user's taste preference. Therefore, the embodiment of the present application can screen the candidate food set by integrating the user's taste taboos (caused by health conditions), so that the food or ingredients used for food recommendation can meet the user's health requirement as much as possible. At the same time, by comparing the similarity between the food and the preferred food, the finally selected candidate food can further meet the user's personalized requirement.

As shown in FIG. 5, optionally, between S510c and S510d, the step S510 can further comprise a step S510f: updating the initial candidate food set based on the taste taboo and the nutritional claim of each food in the initial candidate food set.

Specifically, after obtaining the nutritional claim of the food, the nutritional claim of each food in the initial candidate food set (list) is compared with the taste taboo caused by, for example, the user's health status. If the nutritional claim of a certain food contains inedible nutritional claim in the user's taste taboo, the food will be removed from the initial candidate food list. If the nutritional claim of the food does not contain inedible nutritional claim in the user's taste taboo, the food will be retained in the initial candidate food list to form the final initial candidate food list. For example, when the user's taste taboo is not suitable for eating foods with high sugar content due to diabetes, foods with nutritional claims corresponding to rich sugar and/or high glycemic need to be removed from the initial candidate food list.

As shown in step S510f, after the initial candidate food set is screened by using taste taboo alone, the nutritional claims of all ingredients can be further filtered by using taste taboo to update the initial candidate food set, so as to ensure that the finally selected initial candidate food set contains neither the user's taboo food nor the inedible nutritional claims.

As shown in FIG. 6, the steps S520 (generating a candidate recipe based on the candidate food) as shown in FIG. 5 may comprise:

S520a, obtaining a recipe combination strategy;

S520b, generating a candidate recipe based on the recipe combination strategy and the candidate food.

In general, the recipe combination strategy can be a combination of food types included in the recipe to be recommended (e.g., any one or more meals in the user's breakfast, lunch and dinner in a day). For example, the recipe combination strategy corresponding to breakfast can include eggs, milk and grains, the recipe combination strategy corresponding to lunch can include staple food, meat dishes, vegetarian dishes, soup and fruit, and the recipe combination strategy corresponding to dinner can include staple food, meat dishes, vegetarian dishes, yogurt and nuts.

The recipe combination strategy can be formulated or set in advance according to the authoritative dietary and nutritional standards, such as the Dietary Guidelines for Chinese Residents, so as to ensure the nutritional balance. For example, it can include the matching of each meal or three meals in a day of cereals, vegetables, dairy products, meat, fruits, aquatic products, bean products, nuts, etc. Therefore, the pre-specified recipe combination strategy can be obtained directly from the database storing the strategy.

In some embodiments, the recipe combination strategy can also be obtained according to the user's taste requirements (such as taste preferences or taboos and eating habits). For example, if the user likes Chinese breakfast, the preset recipe combination strategy corresponding to breakfast can be set as porridge, vegetables, etc., or soybean milk, eggs, fried dough sticks, etc.; if the user likes western breakfast, the preset recipe combination strategy corresponding to breakfast is eggs, milk, etc. Alternatively, the preset recipe combination strategy can also be determined through big data query, for example, being set according to the overall eating habits of people in the user's country or region.

In some embodiments, the recipe combination strategy can be determined according to both dietary nutrition standards such as The Dietary Guidelines for Chinese Residents and user's taste requirements, so as to meet personalized taste requirements to a greater extent while achieving dietary nutrition balance. That is, on the basis of the preset recipe combination strategy, improvement is made based on user taste requirements so as to obtain an improved recipe combination strategy. Therefore, the preset recipe combination strategy provided in the present application (such as the preset food matching scheme for three meals in a day) can not only meet the dietary nutritional requirements, but also meet people's daily eating habits, so as to achieve the goal of meat and vegetable matching and balanced nutrition.

After obtaining the recipe combination strategy, the candidate recipe can be generated by combining the strategy with the candidate food. Specifically, food labels can be made according to the type of food. For example, food labels of rice, steamed bread, flower rolls, cakes, and the like are staple foods; food labels corresponding to foods containing more meat, such as Sweet and Sour Spare Ribs, are meat dishes; food labels corresponding to foods cooked with vegetables, such as the mashed eggplant with minced garlic, are vegetable dishes. In this way, the combination of food types in the preset recipe combination strategy can be expressed as a set of food labels. For example, in the breakfast recipe combination strategy, the set of food labels can be {eggs, milk and grains}, and in the lunch recipe combination strategy, the set of food labels can be {staple food, meat dish, vegetable dish, soup and fruit}. Thus, the food that conforms to each label (food type) in the food label set contained in the recipe combination strategy can be selected from the candidate food, so as to obtain the candidate recipe.

In this way, the candidate recipes generated based on the candidate food and recipe combination strategy can not only ensure the nutritional balance, but also take into account of the user's taste preferences and taboos, so as to be in line with the user's eating habits, thereby achieving the purpose of personalized, healthy and accurate recipe recommendation to a certain extent.

As shown in FIG. 6, the step S530 (quantifying weights of candidate ingredients in the candidate recipe based on the energy requirement of the user) as shown in FIG. 5 may comprise S530a-S530c.

At S530a, determining standard weights of nutrients required by the user according to preset energy supply percentages based on the energy requirement of the user, the nutrients including fat, protein and carbohydrate.

In some embodiments, as stated in step S320c, the user energy requirement calculated based on the user's attribute information and physical activity can be the total energy requirement of the user in a day, for example, it is recorded as Energyₜ. Therefore, when recommending recipes, it is necessary to determine the energy requirement of each meal according to the energy distribution proportion of three meals. For example, the energy distribution proportion of three meals in a day can be determined in advance according to the dietary energy distribution information provided by big data or according to the authoritative dietary energy distribution standard. In this way, according to the total energy required by the user in a day and the corresponding energy supply ratio of three meals, the energy intake of each meal of an individual's three meals in a day can be calculated, that is, the energy requirement of each meal. For example, the energy requirement of each meal can be calculated by the following formula (4):

Energyₜ=Energy₁+Energy₂+Energy₃ (4)

Wherein, Energyₜ represents the total energy required by the user in a day, Energy₁, Energy₂, Energy₃ respectively represent energy required for breakfast, lunch and dinner.

If w₁, w₂ and w₃ would respectively represent the proportion of energy required for three meals in the total energy of a day, then Energy₁=Energyₜ*w₁, Energy₂=Energyₜ*w₂, Energy₃=Energyₜ*w₃. For example, the energy distribution proportion of three meals can be: w₁:w₂:w₃=3:4:3, i.e. w₁=30%, w₂=40%, w₃=30%. In this way, by means of the proportional distribution of the energy required for three meals of breakfast, lunch and dinner, the energy required for each of the three meals can be obtained, i.e., the energy supply requirement corresponding to each candidate recipe can be obtained.

After determining the energy required for each meal, the total energy of each meal can be divided into the energy supply standard values of nutrients according to the energy supply percentage of various nutrients in the diet, such as Energy_{f}, Energyₚ and Energy_{c}, which represent the energy supply standard values of fat, the energy supply standard values of protein and the energy supply standard values of carbohydrate respectively, as shown in the following formula (5):

Energy=Energy_{f}+Energyₚ+Energy_{c} (5)

Alternatively, nutrients may also include, for example, various vitamins, minerals (such as sodium) and the like in addition to fats, proteins and carbohydrates. The functional percentage of various nutrients can be set according to national or international standards. For example, according to the dietary habits and characteristics of Chinese people, the energy supply proportion of the three nutrients can be: 55%˜60% of total energy for carbohydrates; 25%˜30% of total energy for fats; 10%˜15% of total energy for proteins.

After obtaining the energy supply standard values of nutrients (e.g., Energy_{f}, Energyₚ and Energy_{c}), it is also necessary to convert them into the standard weight of various nutrients corresponding to the standard values of energy supply (heat). Generally, the conversion between energy (heat) and weight can be realized by using the respective conversion coefficients of various nutrients. For example, the above calculation can be realized by the following formula (6):

$$\{ \begin{matrix}{Weight}_{f} = \frac{{Energy}_{f}}{{θ}_{f}} \\ {Weight}_{p} = \frac{{Energy}_{p}}{{θ}_{p}} \\ {Weight}_{c} = \frac{{Energy}_{c}}{{θ}_{c}}\end{matrix} \qquad ( 6 )$$

In formula (6), Weight_{f}, Weightₚ, Weight_{c} are the standard weights of fat, protein and carbohydrate required in each meal respectively, the unit is g, θ_{f}, θₚ, θ_{c}, represent the conversion coefficient between the standard weight of fat, protein and carbohydrate and the standard value of energy supply respectively, the unit is kcal/g or kJ/g.

At S530b, the nutrient content of each candidate ingredient in the candidate recipe is obtained.

In some embodiments, the nutrient content of various ingredients, especially the content of fat, protein and carbohydrate, can be directly queried according to the table of ingredient, food or food nutritional composition given by the authority. The nutrient content refers to the weight percentage of the nutrient in the ingredient, that is, the number of grams of nutrients per 100 g of ingredients.

At S530c, calculating the weight of each candidate ingredient in the candidate recipe based on the standard weights of nutrients and the nutrient content of each candidate ingredient in the candidate recipe.

In some embodiments, a nutrient weight equation can be constructed based on the nutrient standard weight and the nutrient content of each candidate ingredient in the candidate recipe, and the weight of each candidate ingredient can be obtained by solving the equation. For the purpose of illustration, in some embodiments, the recipe recommendation described in the present application refers to the recipe recommendation based on meal, that is, the recommended recipe is a recipe for one meal. Therefore, for example, the present application analyzes the nutrient weight equation in meals to obtain the weight of each candidate ingredient in each meal.

In some embodiments, the weight of each ingredient in the candidate recipe can be calculated by solving the following formula (7), namely the nutrient weight equation set:

$$\{ \begin{matrix}{fat}_{1} * {x}_{1} + {fat}_{2} * {x}_{2} + . . . + {fat}_{m} * {x}_{m} = {Weight}_{f} \\ {protein}_{1} * {x}_{1} + {protein}_{2} * {x}_{2} + . . . + {protein}_{m} * {x}_{m} = {Weight}_{p} \\ \begin{matrix}{carbohydrate}_{1} * {x}_{1} + {carbohydrate}_{2} * {x}_{2} + . . . + \\ {carbohydrate}_{m} * {x}_{m} = {Weight}_{c}\end{matrix}\end{matrix} \qquad ( 7 )$$

In the formula (7), m is the number of candidate ingredients in the candidate recipe, x, is the weight of the ith ingredient, fatᵢ is the fat content of the ith ingredient, prat n is the protein content of the ith ingredient, carbohydrateᵢ is the carbohydrate content of the ith ingredient, wherein i=1, . . . , m; Weight_{f}, Weightₚ, Weight_{c} respectively represent the standard weight of fat, protein and carbohydrate required by the user. Formula (7) can represent the nutrient weight equation of any meal of breakfast, lunch and dinner. The significance of the three equations in the nutrient weight equation set of formula (7) is that the total weight of a nutrient (fat, protein and carbohydrate) contained in various ingredients in the candidate recipe should be equal to the standard weight value of the nutrient that the user should ingest.

As shown in formula (7), the nutrient weight equation set is a linear equation set (i.e. multivariate first order equation set). Therefore, the solving method of nutrient equation set (i.e. formula (7)) can be discussed in three cases:

(1) When the number of unknowns in the equation set is equal to the number of effective equations (e.g. m=3), the unique solution can be obtained by directly solving the inverse matrix;

(2) When the number of unknowns in the equation set is greater than the number of effective equations (e.g. m>3), the equation set has infinite solutions, and various optimization algorithms can be used to find the optimal solution of the equation set.

(3) When the number of unknowns in the equation set is less than the number of effective equations (e.g. m<3), the least square method can be used to calculate the approximate solution of the equation set.

FIG. 8 shows an example process of the step S530c as shown in FIG. 5.

In some embodiments according to the present disclosure, for the above case (2) (the number of unknowns in the equation set is greater than the number of effective equations), for example, m>3, it can be solved by an optimization algorithm such as particle swarm optimization algorithm.

As shown in FIG. 8, the step S530c (i.e., calculating a weight of each candidate ingredient in the candidate recipe based on the standard weights of nutrients and the nutrient content of each candidate ingredient in the candidate recipe) may comprise:

S810, obtaining a scoring function of the solution of the nutrient equation set;

S820, determining, by an optimization algorithm, a value of the independent variable corresponding to the minimum value of the scoring function as an optimal solution of the nutrient equation set.

For example, the scoring function of the solution of the nutrient equation set can be obtained by the following formula (8):

$$Score = \frac{\vert \overline{{W}_{f}} - {Weight}_{f} \vert}{{Weight}_{f}} + \frac{\vert \overline{{W}_{p}} - {Weight}_{p} \vert}{{Weight}_{p}} + \frac{\vert \overline{{W}_{c}} - {Weight}_{c} \vert}{{Weight}_{c}} \qquad ( 8 )$$

Wherein, Score represents the dependent variable of the scoring function, Weight_{f}, Weightₚ, Weight_{c} respectively represent the standard weights of fat, protein, carbohydrate required by the user; W̅_{f}, W̅ₚ, W̅_{c} are respectively defined by the following formula (9):

$$\{ \begin{matrix}\overline{{W}_{f}} = {fat}_{1} * {x}_{1} + {fat}_{2} * {x}_{2} + . . . + {fat}_{m} * {x}_{m} \\ \overline{{W}_{p}} = {protein}_{1} * {x}_{1} + {protein}_{2} * {x}_{2} + . . . + {protein}_{m} * {x}_{m} \\ \begin{matrix}\overline{{W}_{c}} = {carbohydrate}_{1} * {x}_{1} + {carbohydrate}_{2} * \\ {x}_{2} + . . . + {carbohydrate}_{m} * {x}_{m}\end{matrix}\end{matrix} \qquad ( 9 )$$

The scoring function in formula (8) is a function of m variables, wherein the independent variables are x₁, x₂, . . . , xₘ, and the dependent variable is Score. Since the meaning of the scoring function represents the standardization difference between the total weight of fat W̅_{f}, the total weight of protein W̅ₚ, the total weight of carbohydrate W̅_{c} in various ingredients in the candidate recipe corresponding to any solution of the equation set with their respective standard Weight_{f}, Weightₚ, Weight_{c}, it is used to characterize the possibility that the solution is called the optimal solution. It should be noted that based on formulas (8) and (9), in the scoring function: the greater the score, the greater the difference between the nutrient weight provided by the candidate recipe and the standard weight; the smaller the score, the smaller the difference between the nutrient weight provided by the candidate recipe and the standard weight. Therefore, the smaller the score, the values of the corresponding independent variables x₁, x₂, . . . , xₘ are more likely to be the optimal solution of the equation set. In this way, the minimum value of the scoring function (Score) can be calculated by the optimization algorithm to determine the corresponding optimal solution.

Due to the different weight distribution of fat, protein and carbohydrate, it is necessary to standardize each nutrient respectively in order to better measure the size of the nutrient weight difference. Moreover, the present application proposes not to use the cosine distance as the scoring function because the cosine distance is more a measure of the difference in the direction of two vectors, whereas the present application needs to measure the size difference of each dimension of two vectors.

It should be noted that particle swarm optimization algorithm can be used as the optimization algorithm. The particle swarm optimization algorithm is one of swarm intelligence algorithms, also known as bird swarm foraging algorithm. The idea comes from simulating that birds will share food location information with each other in the process of foraging, so as to quickly find food location. The particle swarm optimization algorithm seeks the global optimal value by a group of particles constantly following the current optimal value. It has the advantages of simple algorithm and fast operation speed. The disadvantage is that it is easy to fall into the local optimal value for multi-modal function or large-scale optimization. However, the nutrient weight equation to be analyzed in the present application is a multivariate first order equation, and there is no high-order unknown. Therefore, it is not a multi-modal function and has a small scale. It is suitable to use particle swarm optimization algorithm for analysis. Alternatively, the minimum value of the scoring function can also be determined by other optimization algorithms, such as genetic algorithm, ant colony algorithm, etc.

In some embodiments, during specific calculation, the particle swarm optimization algorithm can preset the variation range of the independent variable of the scoring function, that is, the weight threshold of various candidate ingredients, for example, it can be set according to the specific conditions of candidate recipes and corresponding candidate ingredients. This ensures that the optimal solution is in a reasonable range, and there will be no negative value, super large value or super small value. Therefore, after obtaining the optimal solution of the equation set, it is necessary to further judge whether the optimal solution is within the weight threshold range. If it is within the weight threshold range, the recommended recipe is generated based on the weight of candidate ingredients and candidate recipes corresponding to the optimal solution. Otherwise, if it is not within the weight threshold range, candidate foods can be selected again from the candidate food list to form a new candidate recipe, and the new candidate recipe can be quantified again (i.e. calculating the weight of the new candidate ingredients), and so on until it meets the weight threshold. For example, the weight threshold of candidate ingredients is determined based on at least one of the weight proportion relationship between each ingredient type in the candidate recipe, the maximum weight and the minimum weight.

FIG. 9 shows a structural block diagram of a recipe recommendation device according to some embodiments of the present disclosure. The recipe recommendation device 900 as shown in FIG. 9 can correspond to the recipe recommendation system 100 as shown in FIG. 1. As shown in FIG. 9, the recipe recommendation device 900 can comprise an obtaining module 910, a determination module 920, and a recommendation module 930.

The obtaining module 910 can be configured to obtain characteristic information of a user, the characteristic information including static characteristic information and dynamic characteristic information of the user. The determination module 920 can be configured to determine a user requirement based on the characteristic information of the user. The recommendation module 930 can be configured to determine a recommended recipe based on the user requirement.

In the recipe recommendation device according to some embodiments of the present disclosure, the user's various requirements (e.g., including taste requirement and energy requirement) on diet are obtained based on the static characteristic information and dynamic characteristic information of the user, and the recommended recipe is determined based on the user to requirements. Hence, in the recipe recommendation method, by comprehensively considering the user's multi-dimensional personal characteristic information (static characteristic information such as user attribute information and dynamic characteristic information such as physical activity and real-time physical state), the user's personalized and accurate dietary requirements are fully reflected, and by determining recipes based on various user requirements (such as subjective taste requirements and objective energy requirements), healthy, refined and diversified food or recipe recommendations are realized.

It should be appreciated that the units or modules recited in the above device correspond to the steps in the method described with reference to FIG. 3. Thus, the operations and features described above with respect to the method are also suitable for the above device and the units contained therein, which will not be repeated here. The above device can be implemented in the browser of the computing device or other security projects in advance, and can also be loaded into the browser of the computing device or its security project by downloading or the like. The corresponding units in the above device can cooperate with the units in the computing device so as to implement the solutions of the embodiments of the present disclosure.

For several modules or units mentioned in the above detailed description, this division is not mandatory. In fact, according to the implementation of the present disclosure, the features and functions of two or more modules or units described above can be embodied in one module or unit. Conversely, the feature and function of one module or unit described above can be embodied by a plurality of modules or units by further division.

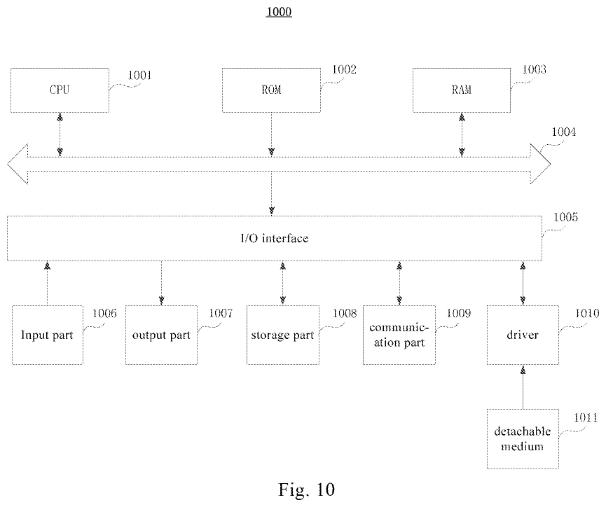

FIG. 10 schematically shows an example block diagram of a computing device 1000 according to some embodiments of the present disclosure. The computing device 1000 can represent a device for implementing various means or modules described herein and/or carrying out various methods described herein. The computing device 1000 can be for example a server, a desktop computer, a laptop computer, a tablet computer, a smart phone or any other appropriate computing devices or computing systems, which can include devices of various levels from full resource devices with large storage and processing resources to low resource devices with limited storage and/or processing resources. In some embodiments, the recipe recommendation device 900 described above with respect to FIG. 9 can be implemented in one or more computing devices 1000 respectively.

As shown in FIG. 10, the computing device can comprise a central processing unit (CPU) 1001, which can execute various appropriate actions and processing based on the programs stored in a read-only memory (ROM) 1002 or the programs loaded from a storage part 1008 to a random access memory (RAM) 1003. In RAM 1003, various programs and data required by system operations are also stored. The CPU 1001, the ROM 1002 and the RAM 1003 are connected to each other through a bus 1004. An input/output (I/O) interface 1005 is also connected to the bus 1004.

The following components are connected to the I/O interface 1005: an input part 1006 including keyboard, mouse, etc.; an output part 1007 including such as a cathode-ray tube (CRT), a liquid crystal display (LCD) and a loudspeaker, etc.; a storage part 1008 including hard disk or the like; and a communication part 1009 including network interface cards such as LAN card, modem, etc. The communication part 1009 performs communication processing via networks such as Internet. A driver 1010 is also connected to the I/O interface 1005 as needed. A detachable medium 1011, such as a magnetic disc, an optical disc, a magnetic optical disk, a semiconductor memory and so on, is mounted on the driver 1010 as needed, so that the computer program read from it can be installed into the storage part 1008 as needed.

Particularly, according to an embodiment of the present disclosure, the above process described with reference to the flow charts can be implemented as a computer software program. For example, an embodiment of the present disclosure comprises a computer program product, which comprises a computer program carried on a machine-readable medium, the computer program comprising program codes for carrying out the method as shown in the flow chart. In such an embodiment, the computer program can be downloaded and installed from the network through the communication part 1009, and/or installed from the detachable medium 1011. When the computer program is executed by the central processing unit (CPU) 1001, the above functions defined in the system of the present disclosure are performed.

It should be noted that the computer readable medium as shown in the present disclosure can be a computer readable signal medium or a computer readable storage medium or any combination of the above two. The computer readable storage medium for example can be, but not limited to: electrical, magnetic, optical, electromagnetic, infrared, or semiconductor systems, apparatuses or devices, or any combination of the above. The more specific examples of the computer readable storage medium can include but not limited to: an electrical connection with one or more conductors, a portable computer disk, a hard disk, a random access memory (RAM), a read only memory (ROM), a erasable programmable read only memory (EPROM or a flash memory), an optical fiber, a portable compact disk read only memory (CD-ROM), a light storage device, a magnetic storage device, or any appropriate combination of the above. In the present disclosure, the computer readable storage medium can be any tangible medium containing or storing programs, which programs can be used by/combined with the instruction execution system, apparatus or device. Moreover, in the present disclosure, the computer readable signal medium can include a data signal that propagates in baseband or as part of a carrier wave, in which computer-readable program codes are carried. Such propagated data signal can take various forms, including but not limited to electromagnetic signal, optical signal or any appropriate combination of the above. The computer readable signal medium can also be any computer readable medium other than the computer readable storage medium. The computer readable medium can send, propagate or transmit programs used by/combined with the instruction execution system, apparatus or device. The program codes contained on the computer readable medium can be transmitted by any appropriate mediums, including but not limited to: wireless, wire, optical cable, RF, etc., or any appropriate combination of the above.

The flow charts and the block diagrams in the drawings illustrate the architectures, functions and operations that may be carried out by the systems, methods and computer program products according to various embodiments of the present disclosure. In this regard, each block in the flow chart or the block diagram can represent a part of a module, a program segment or a code, the part of the module, the program segment or the code comprises one or more executable instructions for implementing the specified logical functions. It shall be also noted that in some alternative implementations, the functions labeled in the blocks may also occur in a sequence different from that labeled in the drawings. For example, two successively represented blocks actually can be performed essentially in parallel, they can also be performed in a reverse order sometimes, which depends on the functions involved. It shall be further noted that each block in the block diagram and/or the flow chart as well as the combination of the blocks in the block diagram and/or the flow chart can be implemented by the dedicated hardware based system for performing the specified functions or operations, or be carried out by the combination of dedicated hardware and the computer instructions.

As another aspect, the present disclosure further provides a computer readable storage medium. The computer readable storage medium can be either a computer readable storage medium comprised in the device described in the above embodiments, or a computer readable storage medium that exists alone without being assembled into the device. The above computer readable storage medium stores one or more programs, which are used by one or more processors for performing the recipe recommendation method described in the present disclosure.

The units or modules described in the embodiments of the present disclosure can be implemented by software or hardware. The units or modules described can also be arranged in a processor. For example, it can be described as: a processor, comprising an obtaining module, a determination module and a recommendation module, etc. The names of these units or modules do not constitute limitation to the units or modules per se under certain circumstances.

The above description is merely preferred embodiments of the present disclosure and explanations on the applied technical principles. Those skilled in the art should understand that the disclosure scope involved in the present disclosure is not limited to the technical solution formed by particular combinations of the above technical features, it should also cover, in the case of not departing from the preceding concept disclosed, other technical solutions formed by any combination of the above technical features or the equivalent features thereof, for example, technical solutions formed by mutual replacement of the above features and technical features disclosed in the present disclosure (but not limited to) with similar functions.

In the description of this Specification, the terms “one embodiment”, “some embodiments”, “an example”, “a specific example”, or “some examples” intend to indicate that the specific features, structure, materials or characteristics described in combination with the embodiment or example are comprised in at least one embodiment or example of the present disclosure. In this Specification, the schematic expressions of the above terms need not be directed to the same embodiment or example. Moreover, the specific features, structure, materials or characteristics described can be combined in an appropriate manner in any one or more embodiments or examples. In addition, in the case of without contradiction, those skilled in the art can combine different embodiments or examples as well as features of different embodiments or examples described in this Specification.

It should be appreciated that the respective parts of the present disclosure can be implemented by hardware, software, firmware or their combinations. In the above implementation, a plurality of steps or methods can be carried out by software or firmware that is stored in the memory and executed by an appropriate instruction execution system. For example, if it is carried out by hardware, it can be carried out by any of the following well known technologies of the art or their combinations: discrete logic circuit with logic gate circuit for realizing logic function on data signal, application specific integrated circuit with suitable combinational logic gate circuit, programmable gate array, field programmable gate array and so on.

The ordinary skilled person in the art can understand that all or part of the steps of the method in the above embodiment can be performed by program instruction related hardware. The program can be stored in a computer readable storage medium. When executed, the program is able to carry out one or a combination of the steps of the method embodiment.

In addition, the functional units in each embodiment of the present disclosure can be integrated in one processing module, or each unit physically exists separately, or two or more units are integrated in one module. The above integrated module can be either implemented in the form of hardware or in the form of a software functional module. The integrated module, if implemented in the form of a software functional module and sold or used as an independent product, can also be stored in a computer readable storage medium.

1. A recipe recommendation method, comprising:
obtaining characteristic information of a user, the characteristic information of the user comprising static characteristic information and dynamic characteristic information of the user;
determining a user requirement based on the characteristic information of the user;
determining a recommended recipe based on the user requirement.

2. The method according to claim 1, wherein the static characteristic information of the user comprises attribute information of the user, the dynamic characteristic information of the user comprises a physical activity of the user within a predetermined time period, and the user requirement comprises an energy requirement of the user.

3. The method according to claim 2, wherein the determining a user requirement based on the characteristic information of the user comprises:
calculating a basal metabolic rate of the user based on the attribute information of the user;
determining a physical activity level coefficient of the user based on the physical activity of the user within a predetermined time period;
calculating the energy requirement of the user based on the basal metabolic rate and the physical activity level coefficient of the user.

4. The method according to claim 3, wherein the determining a physical activity level coefficient of the user based on the physical activity of the user within a predetermined time period comprises:
determining an exercise level of the user based on the physical activity of the user within a predetermined time period;
determining the physical activity level coefficient of the user based on the exercise level of the user.

5. The method according to claim 3, wherein the static characteristic information of the user further comprises taste related information of the user, the dynamic characteristic information of the user further comprises a physical state of the user, and the user requirement further comprises a taste requirement of the user.

6. The method according to claim 5, wherein the determining a user requirement based on the characteristic information of the user further comprises:
obtaining the taste requirement of the user based on the taste related information and the physical state of the user, wherein the taste requirement of the user comprises a taste preference and a taste taboo of the user;
and the determining a recommended recipe based on the user requirement comprises:
determining a recommended recipe based on at least one of the taste requirement and the energy requirement of the user.

7. The method according to claim 6, wherein the determining a recommended recipe based on at least one of the taste requirement and the energy requirement of the user comprises:
determining a candidate food based on the taste requirement of the user;
generating a candidate recipe based on the candidate food;
quantifying weights of candidate ingredients in the candidate recipe based on the energy requirement of the user;
determining a recommended recipe based on the weights of candidate ingredients in the candidate recipe and the candidate recipe.

8. The method according to claim 7, wherein the determining a candidate food based on the taste requirement of the user comprises:
selecting an initial candidate food set from a predetermined food bank based on the taste taboo of the user;
selecting a preferred food for the user from the initial candidate food set based on the taste preference of the user;
determining, for each food in the initial candidate food set, a nutritional claim of the food based on a nutritional claim of main ingredients, a cooking method and a weight proportion of auxiliary ingredients of the food;
calculating, for each food in the initial candidate food set, a similarity between the food and the preferred food based on the nutritional claim, the cooking method and the main ingredients of the food;
selecting a candidate food from the initial candidate food set based on the similarity between each food in the initial candidate food set and the preferred food.

9. The method according to claim 8, wherein the determining a candidate food based on the taste requirement of the user further comprises:
prior to the selecting a candidate food from the initial candidate food set based on the similarity between each food in the initial candidate food set and the preferred food, updating the initial candidate food set based on the taste taboo and the nutritional claim of each food in the initial candidate food set.

10. The method according to claim 8, wherein the nutritional claim comprises a quantifiable nutritional claim and a non-quantifiable nutritional claim; and
the determining, for each food in the initial candidate food set, a nutritional claim of the food based on a nutritional claim of main ingredients, a cooking method and a weight proportion of auxiliary ingredients of the food comprises:
determining whether the weight proportion of auxiliary ingredients in the food exceeds a preset threshold;
in response to the weight proportion of auxiliary ingredients in the food exceeding the preset threshold, obtaining the quantifiable nutritional claim of the auxiliary ingredients and determining the nutritional claim of the food as a nutritional claim of the main ingredients and the quantifiable nutritional claim of the auxiliary ingredients;
in response to the weight proportion of auxiliary ingredients in the food not exceeding the preset threshold, determining the nutritional claim of the food as a nutritional claim of the main ingredients.

11. The method according to claim 7, wherein the generating a candidate recipe based on the candidate food comprises:
obtaining a recipe combination strategy;
generating a candidate recipe based on the recipe combination strategy and the candidate food.

12. The method according to claim 7, wherein the quantifying weights of candidate ingredients in the candidate recipe based on the energy requirement of the user comprises:
determining standard weights of nutrients required by the user according to preset energy supply percentages based on the energy requirement of the user, the nutrients comprising fat, protein and carbohydrate;
obtaining a nutrient content of each candidate ingredient in the candidate recipe;
calculating a weight of each candidate ingredient in the candidate recipe based on the standard weights of the nutrients and the nutrient content of each candidate ingredient in the candidate recipe.

13. The recipe recommendation method according to claim 12, wherein the calculating a weight of each candidate ingredient in the candidate recipe based on the standard weights of the nutrients and the nutrient content of each candidate ingredient in the candidate recipe comprises:
calculating the weight of each candidate ingredient in the candidate recipe by solving the following nutrient weight equation set:
$$\{ \begin{matrix}{fat}_{1} * {x}_{1} + {fat}_{2} * {x}_{2} + . . . + {fat}_{m} * {x}_{m} = {Weight}_{f} \\ {protein}_{1} * {x}_{1} + {protein}_{2} * {x}_{2} + . . . + {protein}_{m} * {x}_{m} = {Weight}_{p} \\ \begin{matrix}{carbohydrate}_{1} * {x}_{1} + {carbohydrate}_{2} * {x}_{2} + . . . + \\ {carbohydrate}_{m} * {x}_{m} = {Weight}_{c}\end{matrix}\end{matrix}$$
wherein in is the number of the candidate ingredients in the candidate recipe, xᵢ is the weight of the ith ingredient, fatᵢ is the fat content of the ith ingredient, proteinᵢ is the protein content of the ith ingredient, carbohydrateᵢ is the carbohydrate content of the ith ingredient, wherein i=1, . . . , m; Weight_{f}, Weightₚ, Weight_{c} respectively represent standard weights of fat, protein, carbohydrate required by the user.

14. The method according to claim 13, wherein the calculating the weight of each candidate ingredient in the candidate recipe by solving the following nutrient weight equation set comprises:
obtaining a scoring function of the solution of the nutrient equation set by the following formula:
$$Score = \frac{\vert \overline{{W}_{f}} - {Weight}_{f} \vert}{{Weight}_{f}} + \frac{\vert \overline{{W}_{p}} - {Weight}_{p} \vert}{{Weight}_{p}} + \frac{\vert \overline{{W}_{c}} - {Weight}_{c} \vert}{{Weight}_{c}} ,$$
wherein Score represents the dependent variable of the scoring function, W̅_{f}, W̅ₚ, W̅_{c} are respectively defined by the following formula:
$$\{ \begin{matrix}\overline{{W}_{f}} = {fat}_{1} * {x}_{1} + {fat}_{2} * {x}_{2} + . . . + {fat}_{m} * {x}_{m} \\ \overline{{W}_{p}} = {protein}_{1} * {x}_{1} + {protein}_{2} * {x}_{2} + . . . + {protein}_{m} * {x}_{m} \\ \begin{matrix}\overline{{W}_{c}} = {carbohydrate}_{1} * {x}_{1} + {carbohydrate}_{2} * \\ {x}_{2} + . . . + {carbohydrate}_{m} * {x}_{m}\end{matrix}\end{matrix} ;$$
determining a value of the independent variable corresponding to the minimum value of the scoring function as an optimal solution of the nutrient equation set by means of an optimization algorithm.

15. The method according to claim 1, wherein the obtaining characteristic information of a user comprises at least one of the following steps:
collecting the characteristic information of the user through human-computer interaction;
receiving the characteristic information of the user from at least one service platform;
collecting the characteristic information of the user through at least one of face monitoring and physical state monitoring.

16. A recipe recommendation device, comprising:
an obtaining module configured to obtain characteristic information of a user, the characteristic information of the user comprising static characteristic information and dynamic characteristic information of the user;
a determination module configured to determine a user requirement based on the characteristic information of the user;
a recommendation module configured to determine a recommended recipe based on the user requirement.

17. A computing device, comprising:
a processor; and
a memory with instructions stored thereon, which instructions, when executed on the processor, cause the processor to carry out the method as claimed in claim 1.

18. A computer readable storage medium, with computer readable instructions stored thereon which, when executed, carry out the method as claimed in claim 1.
